(19) United States
(12) Patent Application Publication
(10) Pub. No.: US 2012/0045431 A1
(43) Pub. Date: Feb. 23, 2012

(54) METHOD OF STABILIZING AND STERILIZING PEPTIDES OR PROTEINS

(75) Inventors: Robert T. Lyons, (US); John T. Trogden, (US)

(73) Assignee: Allergan, Inc.

(21) Appl. No.: 13/211,025

(22) Filed: Aug. 16, 2011

Related U.S. Application Data
(60) Provisional application No. 61/374,529, filed on Aug. 17, 2010.

(51) Int. Cl. A61K 39/395; A61K 38/02; A61L 2/08

(52) U.S. Cl. 424/130.1; 422/22; 514/1.1

(57) ABSTRACT

The present invention provides a method for sterilizing a protein-containing bioerodible implant. The sterilization is accomplished using β-radiation, or high energy electrons. Following sterilization the implant can be used in a variety of methods for the sustained release of a therapeutic protein to treat a disease or condition in a human or non-human subject. The sterilization process is compatible with proteins containing one or more disulfide bonds or easily oxidized methionine residues.

### CROSS REFERENCE TO RELATED APPLICATIONS

This Application claims the benefit of U.S. Provisional Application 61/374,529, filed Aug. 17, 2010, and hereby incorporated by reference.

### BACKGROUND

The present invention relates to a method of stabilizing and/or sterilizing peptides or proteins by irradiating a composite of a peptide and/or a protein incorporated in a polymeric matrix with β radiation.

Common sterilization processes used in the pharmaceutical industry for dry solid materials include dry-heating, steam autoclaving, use of chemical agents (e.g. hydrogen peroxide), use of gaseous agents (e.g. ethylene oxide), and irradiation by ultraviolet light or γ-radiation. But few good options are available for sterilizing proteins in a dry solid state. Dry heat and steam methods involve high heat that destroys protein structure and activity. Chemical agents and ethylene oxide gas also degrade proteins and, in addition, ethylene oxide is carcinogenic. γ-irradiation has high penetrating power and is commonly used for sterilizing medical devices. However, the extended exposure time (hours) to accumulate a sterilizing dose is known to cause localized heating, and traces of water in the dry protein lead to the generation of chemically reactive oxygen radicals. As a result, gamma-irradiation is often associated with irreversible alteration in protein conformation at the molecular level, especially when the protein contains one or more disulfide bonds or easily oxidized methionine residues. In order to prevent radical-induced degradation, antioxidants such as carotenoids or ascorbate are often added to pharmaceutical preparations. It has been reported that L-tyrosine may be used to prevent aggregation during gamma-irradiation of certain proteins in aqueous solution. The stabilization of proteins in solution by polyols such as mannitol, sucrose, glycerol, and lactose has also been studied.

### BRIEF SUMMARY OF THE INVENTION

It has now been discovered that terminal sterilization of freeze-dried therapeutic proteins such as 1121 Fab, a protein available from ImClone, that have been incorporated into a bioabsorbable, biodegradable polymer matrix such as poly(D,L-lactide co-glycolide) (PLGA) may be accomplished by electron beam (β) irradiation, i.e. by high energy electrons or “E-Beam”. (See published US Patent Applications 20090203039 and 200900226399, at paragraphs [0079] and [0047], respectively, for a disclosure of Fab. See, also, “Tailoring in Vitro Selection for a Picomolar Affinity Human Antibody . . . ” 278 The Journal of Biological Chemistry 43496-43507 Oct. 30, 2003 wherein certain uses of 1121 Fab are disclosed.)

In one embodiment, the present invention is directed to a method for sterilizing a biodegradable implant, said implant comprising a polypeptide and a biodegradable polymer matrix, the method comprising
a. mixing said polypeptide with an excipient in aqueous solution to form an admixture, said excipient selected from the group consisting of sucrose, trehalose, and glycine, followed by
b. lyophilizing said admixture, followed by
c. incorporating the lyophilized admixture into a biodegradable polymer matrix, thereby forming a biodegradable implant for the release of said polypeptide in a tissue of a mammalian subject, followed by
d. irradiating the implant with β-radiation, such that the implant receives a dose of β-radiation of between about 1.5 to about 4.0 megarads (Mrad), thereby sterilizing the biodegradable implant.

The polypeptide can be any desired protein, including any therapeutic protein, antibody, or peptide. Incorporating the lyophilized admixture into a biodegradable polymer matrix may comprise blending the admixture with one or more biodegradable polymers, and then extruding (e.g., hot melt extruding) the blend to thereby form the implant. Blending a lyophilized admixture into a biodegradable polymer matrix generally involves blending the dry, lyophilized admixture with one or more dry biodegradable polymers (i.e., a blending of dry powders). The biodegradable polymer matrix may comprise one or more biodegradable polymers selected from the group consisting of methylcellulose, carboxymethylcellulose, hydroxymethylcellulose hydroxypropylcellulose, hydroxyethylcellulose, ethyl cellulose, chitosan, polylactide-co-glycolide (PLGA), polylactic acid (PLA), polyglycolide, polyhydroxybutyric acid, poly(ε-caprolactone), poly(γ-caprolactone), poly(δ-valerolactone), hyaluronic acid, and polyorthoesters. The biodegradable polymer matrix may comprise polymeric microspheres. The microspheres may comprise one or more biodegradable polymers selected from the group consisting of poly(D,L-lactide co-glycolide) and polylactide (PLA).

In a specific embodiment, the biodegradable implant may be sized and configured as an intraocular implant. In another embodiment, the implant may be sized and configured for use as an interarticular implant, as for example one that can be used as a therapeutic implant in a joint to treat a condition, disease, disorder, or inflammation of a joint. The polypeptide may include one or more disulfide bonds or methionine residues. In a specific embodiment, the lyophilized protein admixture comprises 3 to 5% sucrose by weight. The mammalian subject can be a human or non-human mammal, including but not limited to a mouse, rat, rabbit, dog, horse, pig, and guinea pig.

Also within the scope of the present invention is a sterile biodegradable implant comprising a polypeptide, such as an antibody, wherein said implant is produced according to the method described above.

### BRIEF DESCRIPTION OF THE DRAWINGS

FIG. 1 shows the results of Example 1 wherein the specific binding activity of a soluble released protein after irradiation with high energy electrons is measured using ELISA (enzyme-linked immunosorption assay).

FIG. 2 shows the results of Example 1, wherein the total release of non-aggregated protein after irradiation with high energy electrons is measured using SEC-HPLC (size exclusion chromatography).

### DETAILED DESCRIPTION OF THE INVENTION

The following terms as used herein are defined as follows:

“Sterile” means free from living organisms including microorganisms and their spores.

“Biodegradable polymer” means a polymer or polymers which degrade in vivo, and wherein erosion of the polymer or polymers over time occurs concurrently with or subsequent to release of the therapeutic agent. The terms “biodegradable” and “bioerodible” are equivalent and are used interchangeably herein. A biodegradable polymer may be a homopolymer, a copolymer, or a polymer comprising more than two different polymeric units.

“Intraocular implant” means a device or element that is structured, sized, or otherwise configured to be placed “in an eye” of a living mammal, including the subconjunctival space, without causing adverse side effects. In certain embodiments, the intraocular implant may be sized and configured for placement in the vitreous body of the eye, and thereby referred to as an intravitreal implant.

As used herein “a peptide” is a polymer of between about 3 and about 50 contiguous amino acids in length, wherein the amino acids are linked by peptide bonds. In certain embodiments, a peptide is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous amino acids in length. A peptide can be linear, branched, or circular.

As used herein “a polypeptide” means peptides as well as full-length proteins, which proteins may be enzymes or structural proteins, including antibodies (monoclonal or polyclonal). The proteins for use in the present implants may be produced recombinantly or isolated from natural sources.

In the present method for sterilizing peptide and protein implants and polymeric composites using β-irradiation, localized heating is negligible due to very short exposure times at a high dose rate (kGy/sec for β rays compared to kGy/hour for gamma-rays). No residual radioactivity or chemical agents are present after treatment. In the case of proteins that contain disulfide bonds, such as 1121 Fab, it has been discovered that lyophilizing the protein in the presence of sucrose, e.g. 3-5% (w/w) sucrose, prior to adding the protein to the anhydrous polymer matrix (e.g., PLGA) significantly improves protein stability following β irradiation. These results were compared to the stabilizing effects of adding an equal concentration of the disaccharide trehalose, the amino acid glycine, or a “no excipient” control. A higher cumulative release of soluble, non-aggregated protein or of soluble binding activity is suggestive of less denaturation and less formation of insoluble aggregates within the polymer matrix.

Thus, in one embodiment the present invention is directed to a method for sterilizing a protein, such as an antibody, the method comprising mixing said protein with sucrose, in an aqueous solution, to form a protein admixture, lyophilizing said protein admixture, incorporating said lyophilized protein admixture in a bioabsorbable polymer matrix such as poly(D,L-lactide co-glycolide) to form a composite of said protein admixture and said polymer and irradiating said composite with high energy electrons or “E-Beam”, i.e. with β radiation.

The present invention also provides a method of sterilizing proteins, including antibodies, such as 1121 Fab which comprises incorporating said protein in a bioabsorbable polymer matrix such as poly(D,L-lactide co-glycolide) to form a composite of said protein and said polymer and irradiating said composite with β radiation, i.e. high energy electrons or “E-Beam”.

The present invention also provides a method of sterilization of proteins such as 1121 Fab which have been incorporated in a bioabsorbable and biodegradable polymer matrix such as poly(D,L-lactide co-glycolide) to form a composite of said protein and said polymer by irradiating said composite with with β radiation, i.e. high energy electrons or “E-Beam”.

In one aspect of this invention, the composites formed by the method of this invention are used as implants for delivering the protein or peptide to the body of a patient in need thereof. For example, the implant, i.e. an intraocular implant, may be sized accordingly and inserted into the eye of a patient.

In the method of the present invention, a beam of electrons (β-radiation) is used as the radiation source. A beam of electrons has the advantages of high dose rates (e.g., 1, 5, or even 10 Mrad per second), high throughput, less containment, and less confinement equipment. Electron beams can also have up to 80 percent electrical efficiency, allowing for a low energy usage, which can translate into a low cost of operation and low greenhouse gas emissions corresponding to the small amount of energy used. In addition, electrons having energies of 4-10 MeV can have a penetration depth of 5 to 30 mm or more, such as 40 mm, which is more then sufficient to completely sterilize the products of this invention.

Electron beams can be generated, e.g., by electrostatic generators, cascade generators, transformer generators, low energy accelerators with a scanning system, low energy accelerators with a linear cathode, linear accelerators, and pulsed accelerators. Electrons as an ionizing radiation source can be useful, e.g., for relatively thin piles of materials, e.g., less than 0.5 inch, e.g., less than 0.4 inch, 0.3 inch, 0.2 inch, or less than 0.1 inch. In some embodiments, the energy of each electron of the electron beam is from about 0.3 MeV to about 2.0 MeV (million electron volts), e.g., from about 0.5 MeV to about 1.5 MeV, or from about 0.7 MeV to about 1.25 MeV.

The above described sterilized composites of a protein and a biocompatible polymer may be used as implants. For example, the sterilized composites may be sized and structured for use as intraocular implants, intra-articular implants, sub-dermal, or subcutaneous implants. The method of making the implants of the invention is described to illustrate the present invention.

In a first step in the method of the present invention, a protein is incorporated in to a polymeric matrix as disclosed below.

Various techniques may be employed to produce the implants, e.g. the intraocular implants, described herein. Useful techniques include, but are not necessarily limited to, solvent evaporation methods, phase separation methods, interfacial methods, molding methods, injection molding methods, extrusion methods, co-extrusion methods, carver press method, die cutting methods, heat compression, combinations thereof and the like.

Examples of specific methods are discussed in U.S. Pat. No. 4,997,652, incorporated entirely by reference. Extrusion methods may be used to avoid the need for solvents in manufacturing. When using extrusion methods, the polymer and drug (protein) are chosen so as to be stable at the temperatures required for manufacturing, usually at least about 85° C. Extrusion methods use temperatures of about 25° C. to about 150° C., more preferably about 65° C. to about 130° C. An implant may be produced by bringing the temperature to about 60° C. to about 130° C. for drug/polymer mixing, such as about 90° C., for a time period of about 0 to 1 hour, 0 to 30 minutes, or 5-15 minutes. For example, a time period may be about 10 minutes, preferably about 0 to 5 min. The implants are then extruded at a temperature of about 60° C. to about 130° C., such as about 75° C.

In addition, the implant may be coextruded so that a coating is formed over a core region during the manufacture of the implant.

Compression methods may be used to make the implants, and typically yield implants with faster release rates than extrusion methods. Compression methods may use pressures of about 50-150 psi, more preferably about 70-80 psi, even more preferably about 76 psi, and use temperatures of about 0° C. to about 115° C., more preferably about 25° C.

The implants of the present invention may be inserted into the eye, for example the vitreous chamber of the eye, by a variety of methods, including placement by forceps or by trocar following making a 2-3 mm incision in the sclera. The method of placement may influence the therapeutic component or drug release kinetics. For example, delivering the implant with a trocar may result in placement of the implant deeper within the vitreous than placement by forceps, which may result in the implant being closer to the edge of the vitreous.

Among the diseases/conditions which can be treated or addressed in accordance with the present invention include, without limitation, the following: Ocular conditions selected from the group consisting of: macular degeneration, age related macular degeneration, non-exudative age related macular degeneration, exudative age related macular degeneration, choroidal neovascularization, retinopathy, diabetic retinopathy, acute and chronic macular neuroretinopathy, central serous chorioretinopathy, macular edema, cystoid macular edema, and diabetic macular edema, acute multifocal placoid pigment epitheliopathy, Behcet's disease, birdshot retinochoroidopathy, syphilis, lyme disease, tuberculosis, toxoplasmosis, uveitis, intermediate uveitis, pars planitis, and anterior uveitis, multifocal choroiditis, multiple evanescent white dot syndrome, ocular sarcoidosis, posterior scleritis, serpignous choroiditis, subretinal fibrosis, uveitis syndrome, Vogt-Koyanagi-Harada syndrome, retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, central retinal artery occlusion, branch retinal artery occlusion, carotid artery disease, frosted branch angitis, sickle cell retinopathy and other hemoglobinopathies, angioid streaks, familial exudative vitreoretinopathy, Eales disease, sympathetic ophthalmia, uveitic retinal disease, retinal detachment, eye trauma, laser induced eye damage, photocoagulation, eye hypoperfusion during surgery, radiation retinopathy, bone marrow transplant retinopathy, proliferative vitreal retinopathy, appearance of epiretinal membranes, proliferative diabetic retinopathy, ocular histoplasmosis, ocular toxocariasis, presumed ocular histoplasmosis syndrome, endophthalmitis, toxoplasmosis, retinal diseases associated with HIV infection, choroidal disease associated with HIV infection, uveitic disease associated with HIV Infection, viral retinitis, acute retinal necrosis, progressive outer retinal necrosis, fungal retinal diseases, ocular syphilis, ocular tuberculosis, diffuse unilateral subacute neuroretinitis, myiasis, retinitis pigmentosa, systemic disorders with associated retinal dystrophies, congenital stationary night blindness, cone dystrophies, Stargardt's disease and fundus flavimaculatus, Bests disease, pattern dystrophy of the retinal pigmented epithelium, X-linked retinoschisis, Sorsby's fundus dystrophy, benign concentric maculopathy, Bietti's crystalline dystrophy, pseudoxanthoma elasticum, retinal detachment, macular hole, giant retinal tear, retinal disease associated with tumors, congenital hypertrophy of the RPE, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, intraocular lymphoid tumors, punctate inner choroidopathy, acute posterior multifocal placoid pigment epitheliopathy, myopic retinal degeneration and acute retinal pigment epithelitis.

The preferred forms of the polymeric matrix comprise polymeric microspheres, microparticles, microcapsules, or implants. Even more preferred are polymeric microspheres, microparticles, or microcapsules. Most preferably, polymeric microparticles are used in this invention. The term microparticle refers to any polymeric particle having a diameter or equivalent dimension of about 100 micrometers or smaller.

Chemically, the polymeric matrix comprises any polymeric material useful in a body of a mammal, whether derived from a natural source or synthetic. While not intending to be limiting, some examples of useful polymeric materials for the purposes of this invention include carbohydrate based polymers such as methylcellulose, carboxymethylcellulose, hydroxymethylcellulose hydroxypropylcellulose, hydroxyethylcellulose, ethyl cellulose and chitosan; and hydroxy acid polyesters such as polylactide-co-glycolide (PLGA), polylactic acid (PLA), and polyglycolide. Other polymers that may be used in the implants of the present invention include polyhydroxybutyric acid, poly(γ-caprolactone); poly(δ-valerolactone), poly(ε-caprolactone), polycaprolactone, hyaluronic acid, thermal gels and polyorthoesters. Preferably, the polymer of this invention comprises polylactide-co-glycolide (PLGA) or polylactic acid (PLA).

While localized heating is negligible due to very short exposure times at a high dose rate (kGy/sec for β rays it may be desirable to carry out the sterilization step with external cooling.

The term external cooling refers to the use of cooling source on the polymeric material such that the temperature of the polymeric material is lower at the end of the sterilization process than it would be without the external cooling. External cooling of samples during irradiation is widely practiced in the physical, chemical, and biological arts. For example, x-ray crystallography, nuclear magnetic resonance, fluorescence, infrared, microwave, and other such spectroscopic techniques where the sample is irradiated are routinely carried out with external cooling at temperatures ranging from around room temperature to as low as near 0 K. Furthermore, experiments are routinely carried out by practitioners of the chemical and physical arts where samples are irradiated at temperatures ranging from room temperature down to near 0 K. While not intending to limit the scope of the invention in any way, the cooling source could be a bath of a liquid which is cooled by means of a refrigeration method, a cryogenic liquid or solid, or where the liquid is cooled before use. While not intending to limit the scope of the invention in any way, examples of useful cooling baths include ice water, which can cool to temperatures around 0° C.; a dry ice-organic solvent bath, which can cool to temperatures down to about −77° C.; liquid nitrogen, which can cool to temperatures around 77 K; or liquid helium, which can cool to temperatures of 20 K or lower. Alternatively, the cooling source could cool the entire system comprising the radiation source, the polymeric material, and any auxiliary equipment. In such a case, the cooling source could be a cooled room, a freezer or refrigerator. The cooling source could also be cold air from outdoors on a cold day, which could be pumped in, or alternatively, the sterilization could be done outdoors.

The temperature of said polymeric material at the end of the sterilization process is about 10° C. to about 50° C. lower than said temperature would be in the absence of external cooling. For example, the temperature of said polymeric material at the end of the sterilization process is about 20° C. to about 50° C. lower than said temperature would be in the absence of external cooling.

In certain embodiments, the temperature of said polymeric material at the end of the sterilization process is about 50° C. or more lower than said temperature would be in the absence of external cooling.

In other embodiments, sterilization by irradiation is carried out at a temperature below 25° C. For example, the sterilization by irradiation is carried out at a temperature below about 15° C., more preferably, below about 10° C. In another aspect of this invention the sterilization is carried out at a temperature from −25° C. to 5° C.

The term irradiation refers to the process of exposing the composites of this invention to a form of radiation. The type and dose of the radiation used in the irradiation process can be determined by one of ordinary skill in the art by considering the type of polymeric material, the type of any therapeutically active agent that may be present, and the use for which the composite is intended. While not intending to limit the scope of invention, in many cases the dose of the radiation would be similar to that used when sterilizing the sample without external cooling. If the cooling apparatus is comprised of a material that would scatter, reflect, absorb, or otherwise decrease the dose of the radiation received by the sample, the dose should be increased accordingly.

In an aspect of the method of this invention the polymeric material is sterilized by beta irradiation at a dose of about 1.5 to about 4.0 megarads (Mrad), or about 15 kiloGrays (kGy) to about 40 kGy. The polymeric material may be a rod (e.g., extruded filament) or wafer-shaped implant or may be a composition comprising a plurality of biodegradable microspheres. In one embodiment, the polymeric material is sterilized by β-irradiation to a dose of about 15, 16, 17, 18, 19, 20, 21, 22, 23, 24, or 25 kGy, or between about 15 to about 25 kGy.

In certain embodiments of this invention, the polymeric material is used to accomplish the sustained delivery of the therapeutically active agent. The term sustained delivery refers to the delivery of the therapeutically active agent by a system designed to increase its therapeutic half life relative to an identical therapeutically active agent without such a delivery system.

In certain embodiments of the method of the present invention the protein is lyophilized with sucrose prior to incorporation into the polymer matrix.

Lyophilization is a dehydration process which comprises freezing the material(protein) and then reducing the surrounding pressure and adding enough heat to allow the frozen water in the material to sublime directly from the solid phase to the gas phase.

There are three stages in the complete process: freezing, primary drying, and secondary drying.

Freezing is done by placing the material in a freeze-drying flask and rotating the flask in a bath, which is cooled by mechanical refrigeration, dry ice and methanol, or liquid nitrogen, or using a freeze-drying machine. In this step, the protein is cooled below its triple point, the lowest temperature at which the solid and liquid phases of the material can coexist to ensure that sublimation rather than melting will occur in the following steps. Larger crystals are easier to freeze-dry. However, the freezing may be done rapidly, in order to lower the material to below its eutectic point quickly, thus avoiding the formation of ice crystals. Usually, the freezing temperatures are between −50° C. and −80° C.

During the primary drying phase, the pressure is lowered (to the range of a few millibars), and enough heat is supplied to the material for the water to sublimate. The amount of heat necessary can be calculated using the sublimating molecules' latent heat of sublimation. In this initial drying phase, about 95% of the water in the material is sublimated. This phase may be slow, because, if too much heat is added, the material's structure could be altered.

In this phase, pressure is controlled through the application of partial vacuum. The vacuum speeds sublimation, making it useful as a deliberate drying process. Furthermore, a cold condenser chamber and/or condenser plates provide a surface(s) for the water vapor to re-solidify on to prevent water vapor from reaching the vacuum pump, which could degrade the pump's performance. Condenser temperatures are typically below −50° C.

It is important to note that, in this range of pressure, the heat is brought mainly by conduction or radiation; the convection effect is considered to be inefficient.

The secondary drying phase removes unfrozen water molecules, since the ice was removed in the primary drying phase. This part of the freeze-drying process is governed by the material's adsorption isotherms. In this phase, the temperature is raised higher than in the primary drying phase, and can even be above 0° C., to break any physico-chemical interactions that have formed between the water molecules and the frozen material. Usually the pressure is also lowered in this stage to encourage desorption (typically in the range of microbars).

After the freeze-drying process is complete, the vacuum is broken with an inert gas, such as nitrogen, before the material is sealed.

At the end of the operation, the final residual water content in the product is extremely low, around 1% to 4%, by weight.

A person skilled in the art will recognize that there are many ways in which the preferences or embodiments described above can be combined to form unique embodiments. Any combination of the preferences or embodiments mentioned herein which would be obvious to those of ordinary skill in the art are considered to be separate embodiments which fall within the scope of this invention.

The following U.S. Patent Application Publications are hereby incorporated by reference:

US 2005/0003007, filed Jul. 2, 2003, and hereby incorporated by reference, discloses a sterilized polymeric material for use in a body of a mammal wherein said polymeric material is sterilized by irradiation at a reduced temperature.

### EXAMPLES

The following example is intended to illustrate the present invention.

### Example 1

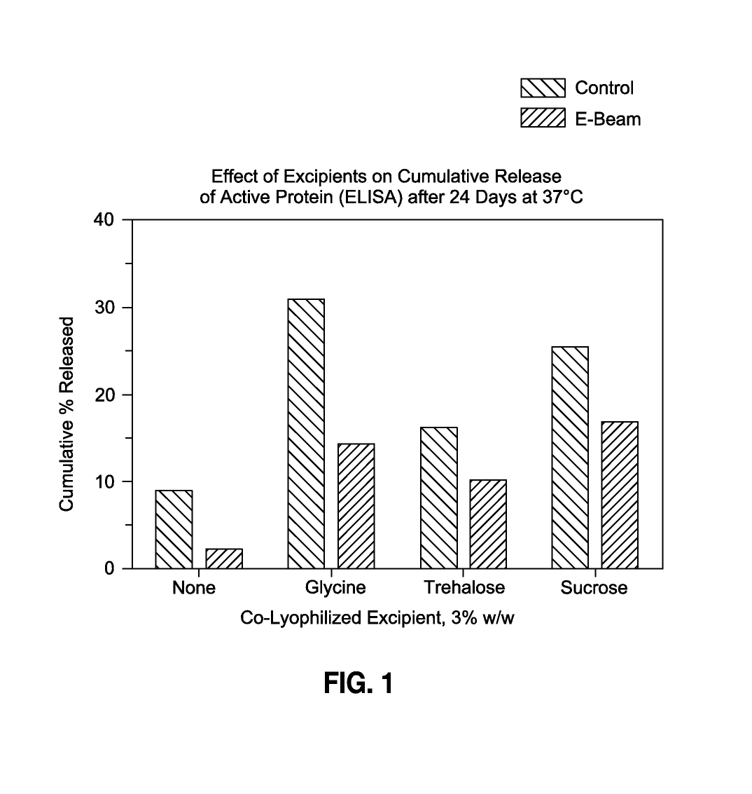

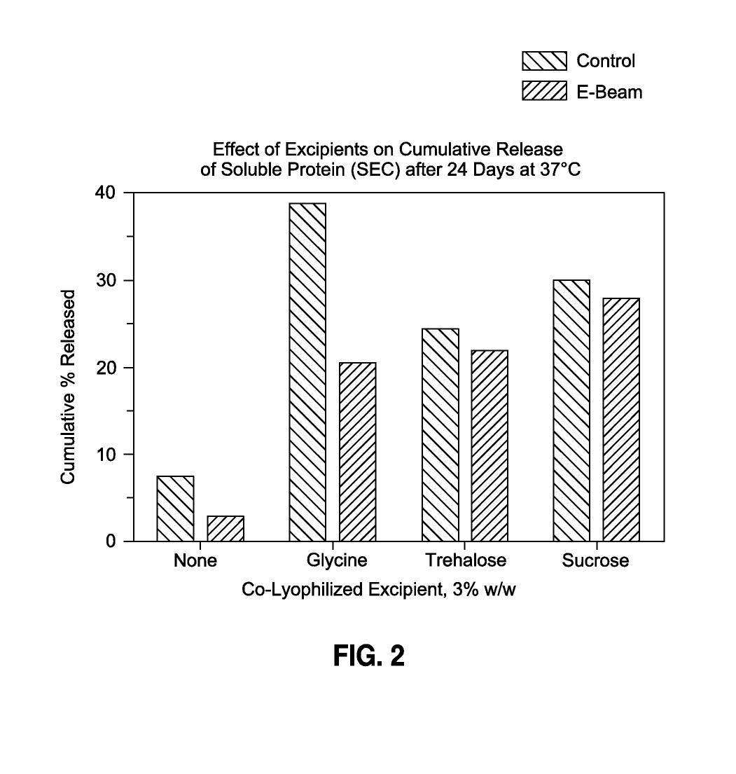

Experimental observations are summarized in FIGS. 1 and 2. SEC-HPLC (size exclusion chromatography) was used to measure the total release of non-aggregated protein. ELISA (enzyme-linked immunosorption assay) was used to monitor the specific binding activity of the released soluble protein in vitro. Under present experimental conditions, a higher cumulative release of soluble, non-aggregated protein or of soluble binding activity is suggestive of less denaturation and less formation of insoluble aggregates within the polymer matrix. The inclusion of the sucrose excipient clearly shows an improvement over the sample containing the glycine excipient and the sample labeled “None”, which does not contain a protective excipient. The sucrose-containing sample also shows an improvement over the sample containing trehelose.

The present invention is not to be limited in scope by the exemplified embodiments, which are only intended as illustrations of specific aspects of the invention. Various modifications of the invention, in addition to those disclosed herein, will be apparent to those skilled in the art by a careful reading of the specification, including the claims, as originally filed. It is intended that all such modifications will fall within the scope of the appended claims. In particular, although the invention is exemplified by Fab 1211, other proteins having therapeutic use in treating the above-described ocular diseases and conditions may be used in the method and composition of this invention. Moreover, peptides, which are short polymers of amino acids linked by peptide bonds and have the same chemical structure as proteins, but are shorter in length, having therapeutic use in treating the above-described ocular diseases and conditions, may be used in the method and composition of this invention.

What is claimed is:

1. A method for sterilizing a biodegradable implant, said implant comprising a polypeptide and a biodegradable polymer matrix, the method comprising
a) mixing said polypeptide with an excipient in aqueous solution to form an admixture, said excipient selected from the group consisting of sucrose, trehalose, and glycine, followed by
b) lyophilizing said admixture, followed by
c) incorporating the lyophilized admixture into a biodegradable polymer matrix, thereby forming a biodegradable implant for the release of said polypeptide in a tissue of a mammalian subject, followed by
d) irradiating the implant with β-radiation such that the implant receives a dose of β-radiation of between about 1.5 to about 4.0 megarads (Mrad), thereby sterilizing the biodegradable implant.

2. The method of claim 1 wherein said polypeptide is an antibody.

3. The method of claim 1, wherein said polypeptide is a peptide.

4. The method of claim 1, wherein the implant is irradiated with β-radiation to a dose of between about 15 kiloGrays (kGy) and about 25 kGy.

5. The method of claim 1, wherein incorporating the lyophilized admixture into a biodegradable polymer matrix comprises blending said admixture with one or more biodegradable polymers, and then extruding the blend to thereby form the implant.

6. The method of claim 4, wherein said one or more biodegradable polymers is selected from the group consisting of methylcellulose, carboxymethylcellulose, hydroxymethylcellulose hydroxypropylcellulose, hydroxyethylcellulose, ethyl cellulose, chitosan, polylactide-co-glycolide (PLGA), polylactic acid (PLA), polyglycolide, polyhydroxybutyric acid, poly(ε-caprolactone), poly(γ-caprolactone), poly(δ-valerolactone), hyaluronic acid, and polyorthoesters.

7. The method of claim 1 wherein said polypeptide includes one or more disulfide bonds or methionine residues.

8. The method of claim 1 wherein said lyophilized protein admixture comprises 3 to 5% sucrose, by weight.

9. The method of claim 1, wherein said biodegradable implant is an intraocular implant.

10. A sterilized intraocular implant produced according to the method of claim 9.

11. A sterilized intraocular implant according to claim 10, wherein said implant comprises a polypeptide selected from the group consisting of an antibody and a peptide.
